(19) United States
(12) Patent Application Publication
(10) Pub. No.: US 2008/0281332 A1
(43) Pub. Date: Nov. 13, 2008

(54) SURGICAL SCREWDRIVER

(75) Inventor: Harold Taylor, Memphis, TN (US)

Correspondence Address: LARSON NEWMAN ABEL POLANSKY & WHITE, LLP, 5914 WEST COURTYARD DRIVE, SUITE 200, AUSTIN, TX, 78730, US

(73) Assignee: WARSAW ORTHOPEDIC, INC., Warsaw, IN (US)

(21) Appl. No.: 11/745,021

(22) Filed: May 7, 2007

(51) Int. Cl. A61B 17/58; A61B 17/86; B25B 25/00; B25B 13/52

(52) U.S. Cl. 606/104; 606/104; 81/52; 81/65.2

(57) ABSTRACT

A surgical screwdriver is disclosed and can include a motor, a microprocessor coupled to the motor, and a key sensor coupled to the microprocessor. The key sensor can be configured to sense a key tag.

### FIELD OF THE DISCLOSURE

The present disclosure relates generally to surgical tools. More specifically, the present disclosure relates to surgical tools used to install surgical screws.

### BACKGROUND

In human anatomy, the spine is a generally flexible column that can take tensile and compressive loads. The spine also allows bending motion and provides a place of attachment for keels, muscles and ligaments. Generally, the spine is divided into three sections: the cervical spine, the thoracic spine and the lumbar spine. The sections of the spine are made up of individual bones (vertebrae) that are separated from each other by intervertebral discs.

The intervertebral discs function as shock absorbers and as joints. Further, the intervertebral discs can absorb the compressive and tensile loads to which the spinal column may be subjected. At the same time, the intervertebral discs can allow adjacent vertebral bodies to move relative to each other a limited amount, particularly during bending, or flexure, of the spine. Thus, the intervertebral discs are under constant muscular and/or gravitational pressure and generally, the intervertebral discs are the first parts of the lumbar spine to show signs of deterioration.

Facet joint degeneration is also common because the facet joints are in almost constant motion with the spine. In fact, facet joint degeneration and disc degeneration frequently occur together. Generally, although one may be the primary problem while the other is a secondary problem resulting from the altered mechanics of the spine, by the time surgical options are considered, both facet joint degeneration and disc degeneration typically have occurred. For example, the altered mechanics of the facet joints and/or intervertebral disc may cause spinal stenosis, degenerative spondylolisthesis, and degenerative scoliosis.

In order to correct certain spinal disorders, it may be necessary to install one or more implants along the spine. For example, scoliosis can be treated using a spinal fixation system. Further, a damaged disc can be replaced using a fusion device, a motion preserving implant, or a similar device. The installation of certain spinal devices may require the use of one or more bone screws to properly position the device and maintain the device in the proper position. Installing bone screws can require great care and improperly installing a bone screw can cause nerve damage and permanent disability to a patient.

Accordingly, there is a need for an improved surgical screwdriver.

### BRIEF DESCRIPTION OF THE DRAWINGS

FIG. 1 is a lateral view of a portion of a vertebral column;

FIG. 2 is a lateral view of a pair of adjacent vertrebrae;

FIG. 3 is a top plan view of a vertebra;

FIG. 4 is a side plan view of a surgical screwdriver in a straight position;

FIG. 5 is a side plan view of a surgical screwdriver in a bent position;

FIG. 6 is a block diagram of a surgical screwdriver system; and

FIG. 7 is a flow chart illustrating one method of using a surgical screwdriver.

### DETAILED DESCRIPTION OF THE DRAWINGS

A surgical screwdriver is disclosed and can include a motor, a microprocessor coupled to the motor, and a sensor coupled to the microprocessor. The key sensor can be configured to sense a key tag.

In another embodiment, a surgical screwdriver is disclosed and can include a housing, a motor within the housing, and a controller within the housing. The controller can be coupled to the motor. The surgical screwdriver can also include a key sensor incorporated in the housing and coupled to the controller.

In yet another embodiment, a method of installing a surgical screw is disclosed and can include retrieving a surgical screw having a key tag and passing the surgical screw near a key sensor incorporated in a surgical screwdriver. The key tag can transmit a maximum number of installation revolutions associated with the surgical screw to a microprocessor within the surgical screwdriver.

In still another embodiment, a kit is disclosed and can include a surgical screwdriver that can have a key sensor and a surgical screw having a key tag.

In another embodiment, a surgical screw is disclosed and can include a shaft and a head coupled to the shaft. The surgical screw can also include a key tag incorporated into the shaft, the head, or a combination thereof. The key tag can be configured to transmit a signal indicating a maximum number of installation revolutions associated with the surgical screw.

### DESCRIPTION OF RELEVANT ANATOMY

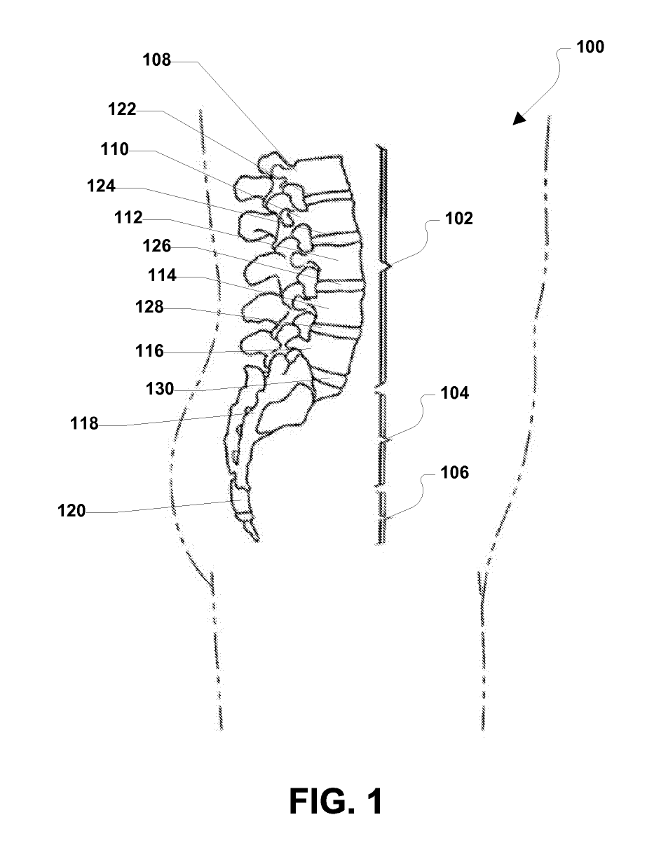

Referring initially to FIG. 1, a portion of a vertebral column, designated 100, is shown. As depicted, the vertebral column 100 includes a lumbar region 102, a sacral region 104, and a coccygeal region 106. As is known in the art, the vertebral column 100 also includes a cervical region and a thoracic region. For clarity and ease of discussion, the cervical region and the thoracic region are not illustrated.

As shown in FIG. 1, the lumbar region 102 includes a first lumbar vertebra 108, a second lumbar vertebra 110, a third lumbar vertebra 112, a fourth lumbar vertebra 114, and a fifth lumbar vertebra 116. The sacral region 104 includes a sacrum 118. Further, the coccygeal region 106 includes a coccyx 120.

As depicted in FIG. 1, a first intervertebral lumbar disc 122 is disposed between the first lumbar vertebra 108 and the second lumbar vertebra 110. A second intervertebral lumbar disc 124 is disposed between the second lumbar vertebra 110 and the third lumbar vertebra 112. A third intervertebral lumbar disc 126 is disposed between the third lumbar vertebra 112 and the fourth lumbar vertebra 114. Further, a fourth intervertebral lumbar disc 128 is disposed between the fourth lumbar vertebra 114 and the fifth lumbar vertebra 116. Additionally, a fifth intervertebral lumbar disc 130 is disposed between the fifth lumbar vertebra 116 and the sacrum 118.

In a particular embodiment, if one of the intervertebral lumbar discs 122, 124, 126, 128, 130 is diseased, degenerated, damaged, or otherwise in need of repair, augmentation or treatment, that intervertebral lumbar disc 122, 124, 126, 128, 130 can be treated in accordance with one or more of the embodiments described herein.

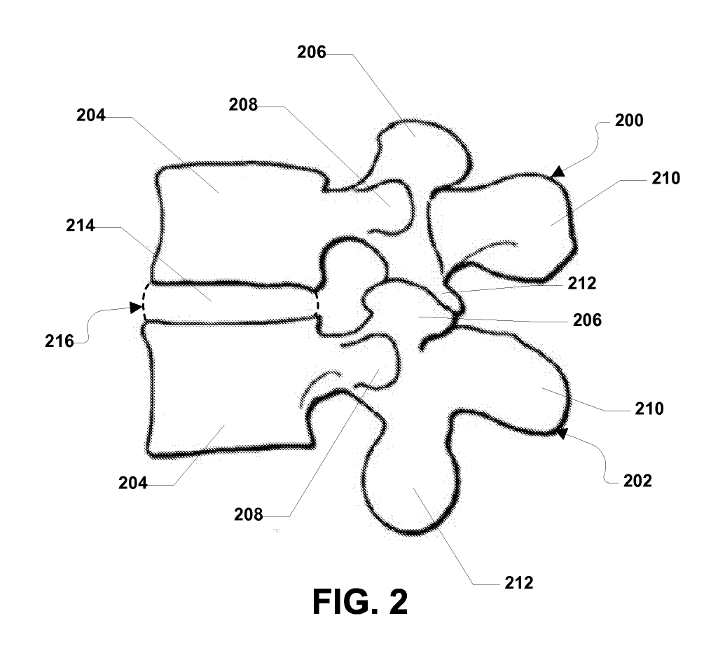

FIG. 2 depicts a detailed lateral view of two adjacent vertebrae, e.g., two of the lumbar vertebra 108, 110, 112, 114, 116 shown in FIG. 1. FIG. 2 illustrates a superior vertebra 200 and an inferior vertebra 202. As shown, each vertebra 200, 202 includes a vertebral body 204, a superior articular process 206, a transverse process 208, a spinous process 210 and an inferior articular process 212. FIG. 2 further depicts an intervertebral disc 216 between the superior vertebra 200 and the inferior vertebra 202.

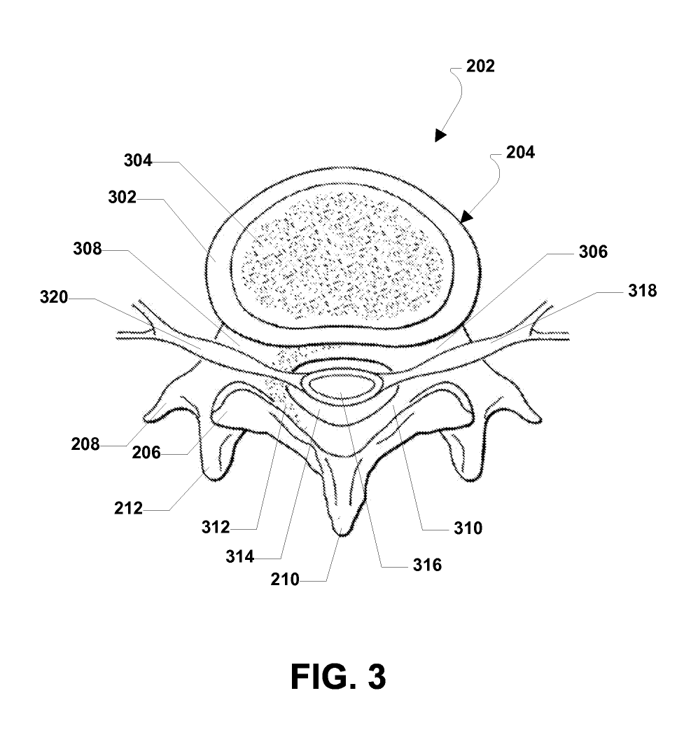

Referring to FIG. 3, a vertebra, e.g., the inferior vertebra 202 (FIG. 2), is illustrated. As shown, the vertebral body 204 of the inferior vertebra 202 includes a cortical rim 302 composed of cortical bone. Also, the vertebral body 204 includes cancellous bone 304 within the cortical rim 302. The cortical rim 302 is often referred to as the apophyseal rim or apophyseal ring. Further, the cancellous bone 304 is softer than the cortical bone of the cortical rim 302.

As illustrated in FIG. 3, the inferior vertebra 202 further includes a first pedicle 306, a second pedicle 308, a first lamina 310, and a second lamina 312. Further, a vertebral foramen 314 is established within the inferior vertebra 202. A spinal cord 316 passes through the vertebral foramen 314. Moreover, a first nerve root 318 and a second nerve root 320 extend from the spinal cord 316.

It is well known in the art that the vertebrae that make up the vertebral column have slightly different appearances as they range from the cervical region to the lumbar region of the vertebral column. However, all of the vertebrae, except the first and second cervical vertebrae, have the same basic structures, e.g., those structures described above in conjunction with FIG. 2 and FIG. 3. The first and second cervical vertebrae are structurally different than the rest of the vertebrae in order to support a skull.

In order to correct certain spinal disorders, it may be necessary to install one or more implants along the spine. For example, scoliosis can be treated using a spinal fixation system. Further, a damaged disc can be replaced using a fusion device, a motion preserving implant, or a similar device. The installation of certain spinal devices may require the use of one or more bone screws to properly position the device and maintain the device in the proper position. The surgical screwdriver described herein may be used to install one or more surgical screws along the spinal column.

### DESCRIPTION OF A SURGICAL SCREWDRIVER

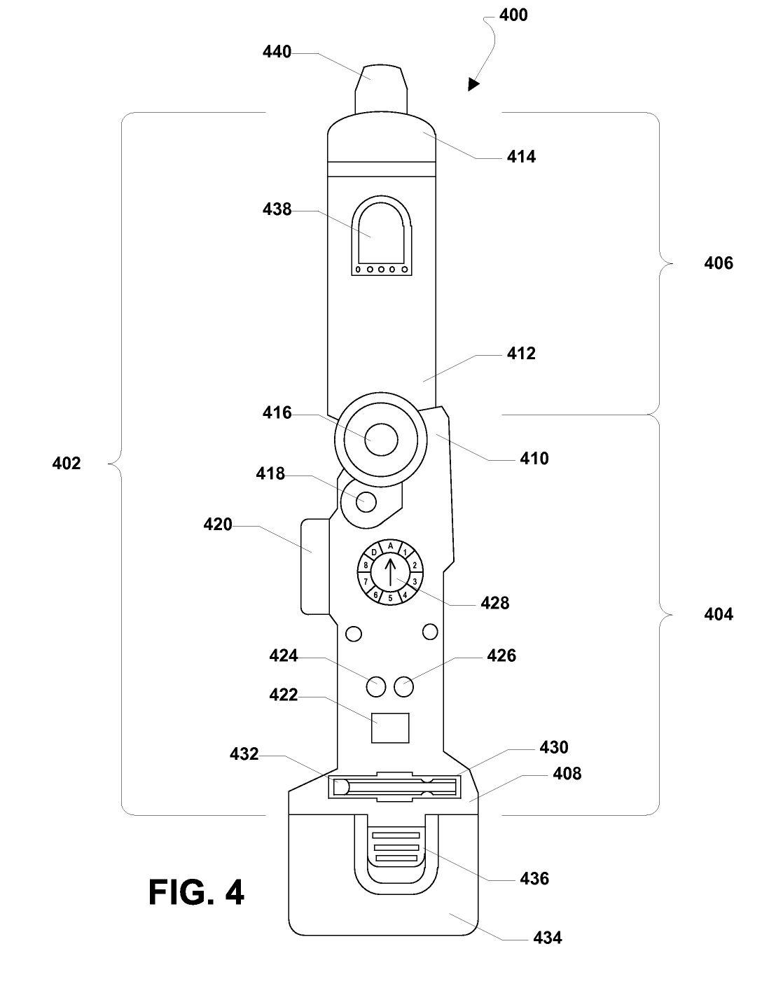

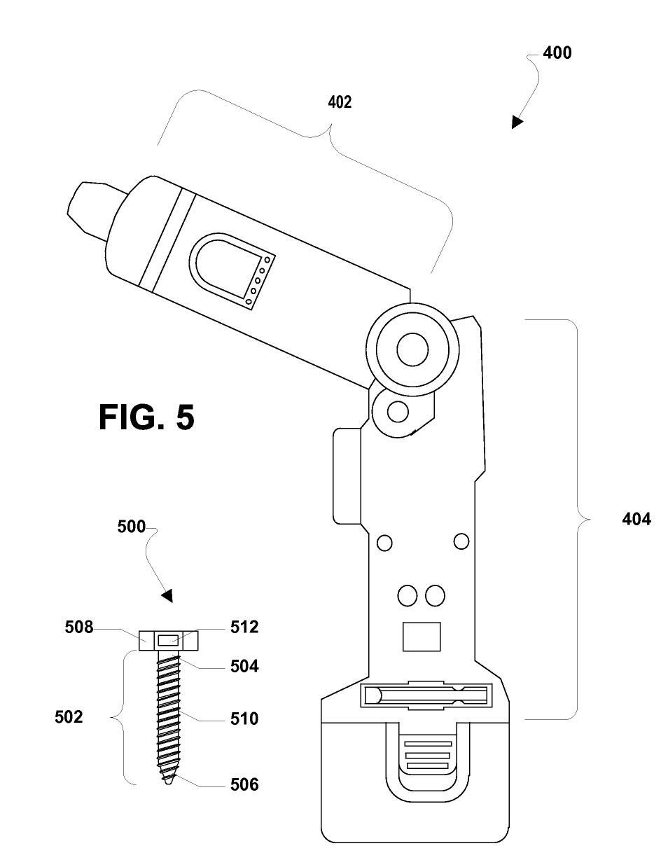

Referring to FIG. 4 and FIG. 5, a surgical screwdriver is shown and is generally designated 400. As shown, the surgical screwdriver 400 can include a housing 402 having a lower portion 404 and an upper portion 406. The lower portion 404 can include a proximal end 408 and a distal end 410. Further, the upper portion 406 can include a proximal end 412 and a distal end 414.

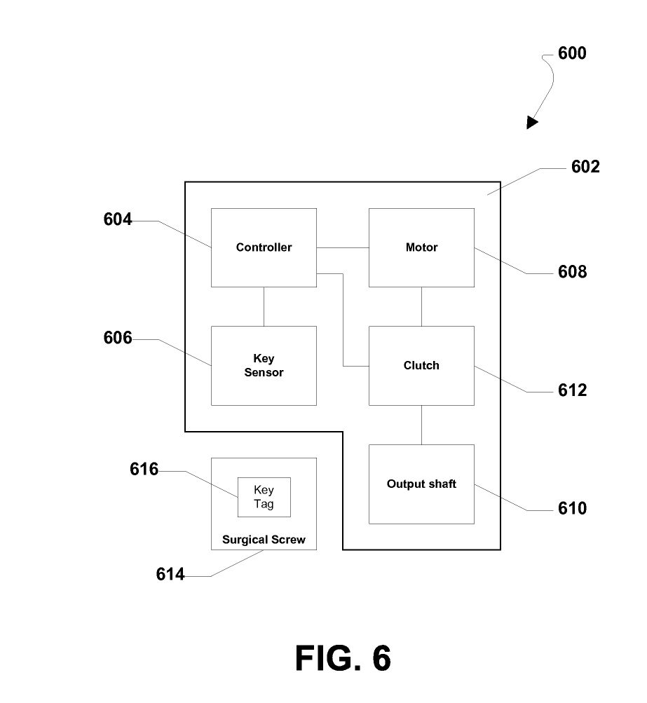

As depicted in FIG. 4, the distal end 410 of the lower portion 404 of the housing 402 can be connected to the proximal end 412 of the upper portion 406 of the housing 402 via a hinge 416. Further, the surgical screwdriver 400 can include a lock 418 that can be incorporated into the distal end 410 of the lower portion 404 of the housing 402 adjacent to the hinge 416. When the lock 418 is pressed, the upper portion 406 of the housing 402 can be rotated relative to the lower portion 404 of the housing 402. As such, the surgical screwdriver 400 is movable between a straight configuration, shown in FIG. 5, and a bent configuration, shown in FIG. 6. In the straight configuration, the upper portion 406 of the housing 402 is substantially aligned with, or coaxial with, the lower portion 404 of the housing 402. In the bent configuration, the upper portion 406 of the housing 402 is angled with respect to the lower portion 404 of the housing 402.

FIG. 4 also indicates that the lower portion 404 of the housing 402 can include a trigger 420 that extends through the lower portion 404 of the housing 402. When the trigger 420 is pressed a motor within the surgical screwdriver 400 is actuated or energized. The lower portion 404 of the housing 402 can also include a key sensor 422 incorporated therein. The key sensor 422 can be configured to sense a key tag attached to a surgical screw, described below. The key tag can be an optical tag, e.g., a bar code tag, a dot code tag, or a combination thereof. The key tag can also be a signal generating tag, e.g., a passive radio frequency identification (RFID) tag, an active RFID tag, or a combination thereof.

In a particular embodiment, the key sensor 422 can be an optical sensor that is configured to sense an optical tag, e.g., a bar code tag, a dot code tag, or a combination thereof. For example, the key sensor 422 can be a bar code sensor. Also, the key sensor 422 can be a dot code sensor. In another embodiment, the key tag can be a signal sensor that is configured to sense a signal generating tag, e.g., a passive RFID tag, an active RFID tag, or a combination thereof. For example, the key sensor 422 can be a Key sensor.

As shown in FIG. 4, the lower portion 404 can include a first indicator light 424 and a second indicator light 426. In a particular embodiment, the indicator lights 424, 426 can be light emitting diodes (LEDs). Further, the indicator lights 424, 426 can indicate whether a key tag placed near the key sensor 422 is sensed. For example, the first indicator light 424 can be a green light that can glow when the key tag placed near the key sensor 422 is sensed. Further, the second indicator light 426 can be a red light that can glow when the key tag placed near the key sensor 422 is not sensed.

In a particular embodiment, a surgical screw having a key tag incorporated therein can be placed in proximity to the key sensor 422. The key sensor 422 can sense the key tag within the surgical screw and transmit a signal to a microprocessor within the surgical screwdriver 400 indicating a maximum number of installation revolutions associated with the surgical screw. The microprocessor can selectively disengaged a clutch within the surgical screwdriver 400 or selectively de-energize a motor within the surgical screwdriver 400 when the maximum number of installations revolutions is reached. It can be appreciated that based on a thread pitch of the surgical screw, the maximum number of installation revolutions can prevent the surgical screw from being advanced too far into the patient. Accordingly, potential damage to the patient is substantially minimized. During use, the indicator lights 424, 426 can indicate to the user whether the surgical screw is properly sensed and identified by the key sensor 422.

FIG. 4 further depicts a dial 428 within the lower portion 404 of the housing 402. The dial 428 can be rotated between a plurality of settings, e.g., automatic (A), one (1), two (2), three (3), four (4), five (5), six (6), seven (7), eight (8), etc. Further, the dial 428 can be rotated to a disable (D) setting. When the dial 428 is rotated to auto (A), the surgical screwdriver 400 can operate as described above, i.e., the key sensor 422 can be used to sense a surgical screw and determine a number of installation revolutions associated with the surgical screw. Alternatively, a surgical screw can be stamped or marked with a number that indicates the number of installation revolutions associated with the surgical screw. A user can rotate the dial 428 to an numerical value around the dial that corresponds to the number that is stamped on the surgical screw and the microprocessor within the surgical screwdriver 400 can prevent the surgical screwdriver 400 from rotating the surgical screw more than the maximum number of installation revolutions, as described herein. When the dial 428 is rotated to disable (D), the surgical screwdriver 400 can operate without the safety feature to prevent over-rotation of the surgical screw. In other words, the surgical screwdriver 400 can operate based on user input received from the trigger.

As shown in FIG. 4, the lower portion 404 of the housing 402 can be formed with a bit pocket 430 and a bit 432 can be removably held therein. In a particular embodiment, the bit 432 can be a straight screwdriver bit, a Phillips screwdriver bit, a star screwdriver bit, a Robertson screwdriver bit, an Allen wrench bit, or any other similar type of tool bit. FIG. 4 also shows a battery 434 that can be removably engaged with the lower portion 404 of the housing 402. In particular, the battery 434 can include a lock 436 that can be slid, or otherwise moved, in order to unlock the battery 434 and allow the battery 434 to be disengaged from the lower portion 404 of the housing 402.

In a particular embodiment, as depicted in FIG. 4, the upper portion 406 of the housing 402 can include a vent 438. The vent 438 can provide airflow to and from a motor within the upper portion 406 of the housing 402. FIG. 4 also shows a chuck 440 extending from the distal end 414 of the upper portion 406 of the housing 402. The chuck 440 can engage a cutting bit, a tool bit, or another type of bit.

FIG. 5 illustrates a surgical screw 500 that can be sensed by the key sensor 422. The surgical screw is shown and is generally designated 500. As shown in FIG. 5, the surgical screw 500 can include a shaft 502 having a proximal end 504 and a distal end 506. A head 508 can be attached to the proximal end 504 of the shaft 502. As shown in FIG. 5, the shaft 502 can include a continuous thread 510 formed along the length of the shaft 502 from the proximal end 504 to the distal end 502 of the shaft 502. FIG. 5 also shows that a key tag 512 can be attached to, or otherwise incorporated into, the head 508 of the surgical screw 500. In another embodiment, the key tag 512 can be attached to, or otherwise incorporated in, the shaft 502 of the surgical screw 500, or in both the head 508 and shaft 502 of the surgical screw 500. In yet another embodiment, the key tag 512 can be attached to, or otherwise incorporated in, the packaging associated with the surgical screw 500, e.g., a box, a bag, or other packaging.

In a particular embodiment, the key tag 512 can indicate a maximum number of installation revolutions associated with the surgical screw 500. For example, to prevent the surgical screw 500 from penetrating too far into the tissue of a patient, the key tag 512 may indicate that the surgical screw 500 has a maximum number of installation revolutions equal to eight. Accordingly, the surgical screw 500 should not be rotated more than eight revolutions. This can substantially prevent the surgical screw 500 from being advanced too far into the patient.

### Description of a Surgical Screwdriver System

Referring now to FIG. 6, a surgical screwdriver system is shown and is generally designed 600. As shown, the system 600 can include a housing 602. A controller 604 can be located within the housing 602. In a particular embodiment, the controller 604 can be an analog controller. Alternatively, the controller 604 can be a digital controller, e.g., a microprocessor.

A key sensor 606 and a motor 608 can be coupled to the controller 604. Further, the system 600 can include a chuck 610 that can be coupled to the motor 608 directly or via a clutch 612. The clutch 612 can also be connected to the controller 604. FIG. 6 further indicates that the system 600 can include a surgical screw 614 having a key tag 616.

In a particular embodiment, the key tag 616 can indicate a maximum number of installation revolutions associated with the surgical screw 614. Further, during use, the surgical screw 614 can be placed in proximity to the key sensor 606. The key sensor 606 can sense the key tag 616 and transmit a signal to a controller 604 to indicate the maximum number of installation revolutions associated with the surgical screw 614. Thereafter, the surgical screw 614 can be engaged with the chuck 610. As the surgical screw 614 is rotated and advanced into a patient, the microprocessor can monitor the revolutions of the motor 608. When the maximum number of installation revolutions is reached, the controller 604 can de-energize the motor 608 to prevent over-rotation of the surgical screw 614. Alternatively, the controller 604 can send a signal to actuate the clutch 612 in order to disengage the chuck 610 from the motor 608 and prevent over-rotation of the surgical screw 614.

### Description of a Method of Using a Surgical Screwdriver

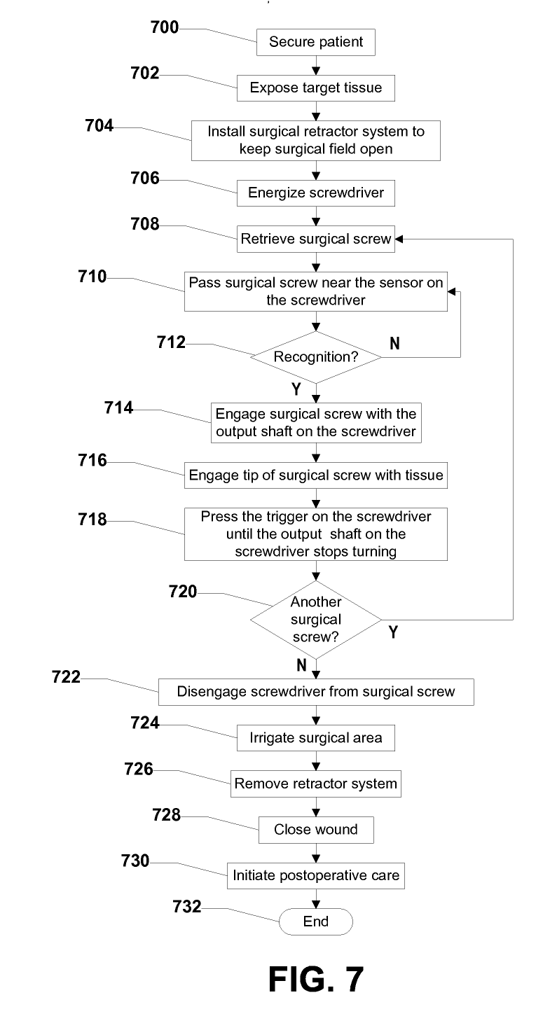

Referring to FIG. 7, a method of using a surgical screwdriver is shown and commences at block 700. At block 700, a patient can be secured on an operating table. For example, the patient can be secured in a prone position to allow a posterior approach to be used to access the patient's spinal column. Alternatively, the patient can be secured in a supine position to allow an anterior approach to be used to access the patient's spinal column. Further, the patient can be secured in a lateral decubitus position to allow a lateral approach to be used to access the patient's spinal column.

Moving to block 702, the target tissue is exposed. Further, at block 704, a surgical retractor system can be installed to keep the surgical field open. For example, the surgical retractor system can be a surgical retractor system configured for posterior access to a spinal column. Alternatively, the surgical retractor system can be a surgical retractor system configured for anterior access to a spinal column. Also, the surgical retractor system can be a surgical retractor system configured for lateral access to a spinal column.

Moving to block 706, the surgical screwdriver can be energized. At block 708, a surgical screw can be retrieved. Thereafter, at block 710, surgical screw can be passed, or placed, near a sensor on the screwdriver, e.g., a key sensor on the screwdriver. Proceeding to decision step 712, the user can determine whether the surgical screwdriver recognized the surgical screw, e.g., by lighting one or more indicator lights on the surgical screwdriver. If the surgical screwdriver does not recognize, or sense, the surgical screw, e.g., a key tag on the surgical screw, the method can return to block 710 and continue as described herein. On the other hand if the surgical screwdriver recognizes the surgical screw, the method can proceed to block 714.

At block 714, the surgical screw can be engaged with a chuck on the surgical screwdriver. Thereafter, at block 716, the tip, or leading end, of the surgical screw can be engaged with tissue of the patient. At block 718, a trigger on the screwdriver can be pressed and held until the chuck on the screwdriver stops turning. Continuing to decision step 720, a user can determine whether to install another surgical screw. If so, the method can return to block 708 and continue as described herein. If another surgical screw is not necessary, the method can proceed to block 722 and the surgical screwdriver can be disengaged from the surgical screw.

Moving to block 724, the surgical space can be irrigated. Further, at block 726, the retractor system can be removed. At block 728, the surgical wound can be closed. The surgical wound can be closed using sutures, surgical staples, or any other surgical technique well known in the art. Moving to block 730, postoperative care can be initiated. The method can end at state 732.

### CONCLUSION

With the configuration of structure described above, the surgical screwdriver provides a device that can be used to install surgical screws within a patient. The surgical screwdriver can substantially prevent a surgical screw from being over-rotated within the patient. Further, the surgical screwdriver can substantially prevent a surgical screw from being over-advanced into the patient. Also, the surgical screwdriver can substantially prevent a surgical screw from being over-tightened within a patient. The surgical screwdriver can be used to place surgical screws within any bony tissue, e.g., along a spinal column, long bones, skull plates, or other bones within a patient.

The above-disclosed subject matter is to be considered illustrative, and not restrictive, and the appended claims are intended to cover all such modifications, enhancements, and other embodiments that fall within the true spirit and scope of the present invention. Thus, to the maximum extent allowed by law, the scope of the present invention is to be determined by the broadest permissible interpretation of the following claims and their equivalents, and shall not be restricted or limited by the foregoing detailed description.

What is claimed is:

1. A surgical screwdriver, comprising:
a motor;
a microprocessor coupled to the motor; and
a key sensor coupled to the microprocessor wherein the key sensor is configured to sense a key tag.

2. The surgical screwdriver of claim 1, wherein the key sensor comprises an optical sensor.

3. The surgical screwdriver of claim 2, wherein the optical sensor comprises a bar code sensor, a dot code sensor, or a combination thereof.

4. The surgical screwdriver of claim 3, wherein the key tag comprises an optical tag.

5. The surgical screwdriver of claim 4, wherein the optical tag comprises a bar code tag, a dot code tag, or a combination thereof.

6. The surgical screwdriver of claim 1, wherein the key sensor comprises a signal sensor.

7. The surgical screwdriver of claim 6, wherein the signal sensor comprises a radio frequency identification (RFID) sensor.

8. The surgical screwdriver of claim 7, wherein the key tag comprises a signal generating tag.

9. The surgical screwdriver of claim 8, wherein the signal generating tag comprises a passive RFID tag, an active RFID tag, or a combination thereof.

10. The surgical screwdriver of claim 1, wherein the microprocessor is configured to selectively control the operation of the motor based on a signal received from the key sensor.

11. The surgical screwdriver of claim 10, wherein the key tag is coupled to a surgical screw.

12. The surgical screwdriver of claim 11, wherein the key tag is configured to transmit a maximum number of installation revolutions associated with the surgical screw.

13. The surgical screwdriver of claim 12, wherein the microprocessor is configured to monitor a number of operating revolutions of the motor.

14. The surgical screwdriver of claim 13, wherein the microprocessor is configured to selectively de-energize the motor when the maximum number of installation revolutions is reached.

15. The surgical screwdriver of claim 13, wherein the microprocessor is configured to selectively disengage a clutch coupled to the motor when the maximum number of installation revolutions is reached.

16. The surgical screwdriver of claim 12, wherein the microprocessor is configured to substantially prevent over-rotation of the surgical screw based on the maximum number of installation revolutions received from the key tag.

17. The surgical screwdriver of claim 12, wherein the microprocessor is configured to substantially prevent over-advancement of the surgical screw based on the maximum number of installation revolutions received from the key tag.

18. The surgical screwdriver of claim 12, wherein the microprocessor is configured to substantially prevent over-tightening of the surgical screw based on the maximum number of installation revolutions received from the key tag.

19. A surgical screwdriver, comprising:
a housing;
a motor within the housing;
a controller coupled to the motor; and
a key sensor incorporated in the housing, wherein the key sensor is coupled to the controller.

20. The surgical screwdriver of claim 19, wherein the key sensor is configured to sense a key tag incorporate in a surgical screw and retrieve data from the key tag.

21. The surgical screwdriver of claim 20, wherein the data from the key tag indicates a maximum number of installation revolutions associated with the surgical screw.

22. The surgical screwdriver of claim 21, wherein the key sensor transmits a signal to the controller indicating the maximum number of installation revolutions associated with the surgical screw.

23. The surgical screwdriver of claim 22, wherein the controller is configured to control the operation of the motor based on a maximum number of installation revolutions associated with a surgical screw.

24. The surgical screwdriver of claim 20, further comprising an indicator incorporated into the housing and coupled to the controller, wherein the indicator is configured to indicate whether a key tag placed near the key sensor is sensed.

25. The surgical screwdriver of claim 19, further comprising a dial, wherein the dial is connected to the controller and wherein the dial is rotated between an automatic setting, at least one numerical setting, and a disable setting.

26. The surgical screwdriver of claim 25, wherein when the dial is rotated to the automatic setting the key sensor, the surgical screwdriver is configured to sense a key tag and automatically control the operation of the motor based on a signal from the key tag.

27. The surgical screwdriver of claim 26, wherein when the dial is rotated to the at least one numerical setting, the surgical screwdriver is configured to control the operation of the motor based on the at least one numerical setting.

28. The surgical screwdriver of claim 27, wherein when the dial is rotated to the disable setting, the surgical screwdriver is configured to control the operation of the motor based on user input received from a trigger coupled to the motor.

29. A method of installing a surgical screw, comprising:
retrieving a surgical screw having a key tag; and
passing the surgical screw near a key sensor incorporated in a surgical screwdriver, wherein the key tag transmits a maximum number of installation revolutions associated with the surgical screw to a microprocessor within the surgical screwdriver.

30. The method of claim 29, further comprising:
engaging the surgical screw with a bit installed in the surgical screwdriver;
engaging a tip of the surgical screw with tissue of a patient; and
pressing a trigger on the surgical screwdriver until the bit automatically stops turning.

31. A kit, comprising:
a surgical screwdriver having a key sensor; and
a surgical screw having a key tag.

32. A surgical screw, comprising:
a key tag incorporated into the surgical screw, wherein the key tag is configured to contain information indicating a maximum number of installation revolutions associated with the surgical screw.
